(19) United States
(12) Patent Application Publication
(10) Pub. No.: US 2011/0151547 A1
(43) Pub. Date: Jun. 23, 2011

(54) REACTOR AND METHOD FOR TREATING CONTAMINATED WATER

(76) Inventors: Konstantin Bloch, PetachTikva (IL); Pnina Vardi, Haifa (IL)

(21) Appl. No.: 13/059,522

(22) PCT Filed: Aug. 18, 2009
(86) PCT No.: PCT/IL2009/000812

§ 371 Date: Feb. 17, 2011

Related U.S. Application Data
(60) Provisional application No. 61/089,790, filed on Aug. 18, 2008.

(51) Int. Cl. A62D 3/02; C12M 1/00

(52) U.S. Cl. 435/262.5; 435/289.1; 435/292.1

(57) ABSTRACT

A reactor and method are presented for processing water, in a manner enabling treatment of contaminated water to produce purified water and enabling simultaneous production of a biomass. The reactor comprises first and second compartments interfacing one another by a gas-permeable membrane. The first compartment serves for interaction between the water and at least one species of aerobic heterotrophic microorganism which are adapted to break down one or more organic contaminants in the contaminated water. The second compartment serves for interaction between the water and at least one species of phototrophic microorganism capable of producing oxygen. The gas-permeable membrane prevents transfer of the organic contaminant therethrough, while allows gas diffusion therethrough. As a result, oxygen, produced by the phototrophic microorganism in the second compartment, is allowed to pass into the first compartment thus facilitating breakage down of the one or more contaminants of the contaminated water in the first compartment.

### FIELD OF THE INVENTION

The present invention relates to the purification of wastewater generated by agricultural, livestock, aqua-culture, industrial or urban activities, as well as to the production of ecologically pure microalgae biomass.

### REFERENCES

The following references are considered to be pertinent for the purpose of understanding the background of the present invention:
1. Aiba, S. Growth kinetics of photosynthetic microorganisms, Adv. Biochem. Eng., 1982, 23, 85-156.
2. Borde, X; Guieysse, B; Delgado, 0; Munoz, R; Hatti-Kaul, R; Nugier-Chauvin, C; Patin, H; Mattiasson, B. Synergistic relationships in algal-bacterial microcosms for the treatment of aromatic pollutants, Bioresourc. Technol, 2003, 86, 293-300
3. Borowitzka, M A. Commercial production of microalgae: ponds, tanks, tubes and fermenters, J. Biotechnol., 1999, 70, 313-321.
4. Brandi, G; Sisti, M; Amagliani, G. Evaluation of the environmental impact of microbial aerosols generated by wastewater treatment plants utilizing different aeration systems. J. Appl. Microbiol. 2000, 88, 845-852.
5. Chen, C. Y. & Lin, J. H. Toxicity of chlorophenols to Pseudokirchneriella subcapitata under air-tight test environment, Chemosphere, 2006, 62, 503-509.
6. Chisti, Y. Biodiesel from microalgae. Biotechnol Adv., 2007, 25, 294-306
7. Cohen, Y. Bioremediation of oil by marine microbial mats. Int Microbiol. 2002, 5, 189-93.
8. de-Bashan, L E; Moreno, M; Hernandez, J P; Bashan, Y. Removal of ammonium and phosphorus ions from synthetic wastewater by the microalgae Chlorella vulgaris coimmobilized in alginate beads with the microalgae growth-promoting bacterium Azospirillum brasilense. Water Res. 2002, 36, 2941-2948.
9. Golueke, C G; Oswald, W J; Gotaas, H B. Anaerobic Digestion Of Algae. Appl Microbiol. 1957, 5, 47-55.
10. Hamoda, M F. Air pollutants emissions from waste treatment and disposal facilities, J. Environ. Sci. Health Part A—Toxic/Hazard. Subst. Environ. Eng. 2006, 41, 77-85.
11. Mallick, N. Biotechnological potential of immobilized algae for wastewater N, P and metal removal: a review. Biometals, 2002, 15, 377-390.
12. Munoz, R; Guieysse, B. Algal-bacterial processes for the treatment of hazardous contaminants: a review. Water Res., 2006, 40, 2799-2815.
13. Muñoz, R; Kollner, C; Guieysse, B; Mattiasson, B. Photosynthetically oxygenated salicylate biodegradation in a continuous stirred tank photobioreactor, Biotechnol. Bioeng,. 2004, 87, 797-803
14. Nicolella, C; van Loosdrecht, M C; Heijnen J J. Wastewater treatment with particulate biofilm reactors. J Biotechnol., 2000, 80, 1-33.
15. Oswald, W J. The coming industry of controlled photosynthesis. Am J Public Health Nations Health, 1962, 52, 235-242.
16. Safonova, E; Dmitrieva, L A; Kvitko, K. V. The interaction of algae with alcanotrophic bacteria in black oil decomposition, Resourc. Conserv. Recycl. 1999, 27, 193-201.
17. Safonova, E; Kvitko, K. V; lankevitch, M. I; Surgko, L. F; A fri I. A; Reisser, W. Biotreatment of industrial wastewater by selected algal-bacterial consortia, Eng. Life Sc. 2004, 4, 347-353.
18. Spolaore, P; Joannis-Cassan, C; Duran, E; Isambert, A. Commercial applications of microalgae. J Biosci Bioeng. 2006, 101, 87-96.
19. Richmond, A. Microalgal biotechnology at the turn of the millennium: a personal view, J. Appl. Phycol., 2000, 12, 441-451.
20. Rothschild, L J; Mancinelli, R L. Life in extreme environments. Nature, 2001, 409, 1092-1101.
21. Wolf, G; Picioreanu, C; van Loosdrecht, M C. Kinetic modeling of phototrophic biofilms: the PHOBIA model. Biotechnol Bioeng. 2007, 97, 1064-1079.

### BACKGROUND OF THE INVENTION

Heterotrophic microorganisms typically utilize organic compounds, toxic for other organisms, as an energy source for their metabolism. These heterotrophic microorganisms are frequently used in a bioremediation process, intended as a pollution control technology, in which the microorganisms break down the organic pollutants in different environments. Typically, the bioremediation of a contaminated site works either by anaerobic or by aerobic biodegradation. Anaerobic biodegradation is the breakdown in an anoxic environment of organic contaminants by microorganisms. The aerobic process takes place in the presence of oxygen. Generally, aerobic microorganisms use oxygen as an electron acceptor, and break down organic chemicals, with the production of carbon dioxide and water. Many toxic compounds are more easily degraded aerobically than anaerobically.

One of the most serious limitations in aerobic biodegradation of waste materials in water is insufficient oxygenation, due to low solubility of oxygen in water. This oxygen deficiency results in microbial growth inhibition and in a decreased rate of toxin degradation by heterotrophic microorganisms such as bacteria and fungi. In order to solve the problem of limited oxygen supply, different technological solutions have been suggested including mechanical aeration and air bubbling.

Several techniques using aerobic reactions and reactors thereof have been developed for treating organic wastes.

For example, U.S. Pat. No. 5,277,814 discloses a method for aerobically treating water-containing organic waste. The process is conducted in a closed reactor with suitable controls to prevent any adverse environmental impact, and involves mixing the wastes with an inert and non-friable bulking agent comprising a substantial component having a density less than water in the presence of an active biomass. An oxygen-containing gas is passed through the reaction mixture to assist in the removal of excess (free) water from the wastes to form wetted high solids content reaction mixture containing the waste solids mixed in a bed of bulking agent. Aerobic reaction conditions are employed to convert the wastes to a treated waste.

U.S. Pat. No. 5,904,851 discloses a process for enriching water with oxygen, where water is conducted through a water inlet into a sealed enriching space and through one or more turbulent mixers in the enriching space, oxygen is introduced through an oxygen inlet into the water in the enriching space before passing through the turbulent mixer, and the oxygen-enriched water is recovered. The invention further includes an aerobic process by carrying out a chemical or microbiological reaction in the oxygen enriched water as the reaction medium, and a therapeutic process of carrying out a therapeutic treatment of a body with an agent containing the oxygen enriched liquid as a vehicle. The invention also concerns as apparatus for enriching water with oxygen, having a water inlet, a sealed enriching space containing an oxygen inlet, and one or more turbulent mixers, and an outlet for oxygenated water.

Some other techniques for wastewater treatment are disclosed for example in U.S. Pat. No. 6,896,804 (this technique utilizes continual introduction of microalgae to produce high amount of oxygen) and U.S. Pat. No. 4,267,038 (this technique utilizes reduction of the waste organics to inorganic forms available for microalgae culture in tanks designed for rapid growth).

### General Description

There is a need in the art in a novel reactor for treating contaminated water and for producing algae biomass.

As used in this specification and the accompanying claims, contaminated water indicates water containing one or more contaminants/impurities at a level that renders the water unsuitable for a specific use (e.g. as drinking water). Generally, contaminants are substances that are either present in an environment (water in this case) where they do not belong or are present at levels that might cause harmful effects to humans or the environment. Contaminants include, but are not limited to, microbiological contaminants such as bacteria, as well as organic contaminants such as, for example, volatile synthetic organic chemicals or VOC's (e.g., hydrocarbons, alcohols, phenols, ethers, acids, sulphides, phosphates, etc.). As used in this specification and the accompanying claims, the term biomass refers to an amount of biological material derived from living, or recently living organisms. In some exemplary embodiments of the invention, biomass is the product, either directly or indirectly, of photosynthesis (the process by which plants use solar energy and atmospheric carbon dioxide to make carbohydrates); Biomass may be produced by algae, being termed algae biomass.

A need for a novel hybrid reactor is associated with the following problems with the known approaches (U.S. Pat. No. 5,277,814; U.S. Pat. No. 5,904,851) for aerobically treating (i.e. generally “using air” where “air usually means oxygen) the water-containing organic waste. Although these techniques are capable of providing an increased level of dissolved oxygen for aerobic biodegradation (i.e. chemical breakdown or decomposition of materials by a physiological environment, e.g. biomineralisation, in which organic matter is converted into minerals), they are costly and can provoke contamination of the atmosphere due to enhanced evaporation of hazardous pollutants [4]; [10].

Another known approach to enhance oxygen supply for wastewater treatment is the use of symbiotic interactions between photosynthetic microalgae and heterotrophic bacteria. The main principles of photosynthetic oxygen supply to heterotrophic bacteria for the efficient aerobic biodegradation of pollutants have been developed by Oswald [9], [15]. The concept of algal bacterial consortium has been intensively studied by various research teams [2]; [7]; [12]; [13]; [16]; [17]. The design of such microbial symbiosis is based on the algal cells' capacity to use light energy for the synthesis of organic molecules from CO₂ and water, and liberate the oxygen required by aerobic bacteria to break down hazardous organic pollutants. In turn, microalgae use CO₂ released during bacterial respiration for photosynthesis.

Prokaryotic and eukaryotic microalgae have undergone remarkable environmental adaptation and are able to liberate oxygen in different extreme conditions of low and high pH, temperature and salinity [20]. The photosynthetic potential of microalgae can be utilized for oxygenation in various technologies requiring aerobic degradation of organic compounds by bacteria.

As indicated above, it is known to utilize microalgae to prevent contamination of atmosphere. However, unicellular algae are much more sensitive to toxic pollutants [5] and their multiplication rate is less intensive compared to bacteria [1]. In addition, algae and bacteria may require different pH and temperature for cultivation. Thus, special attention must be given in selecting optimal partners for such consortia, otherwise the symbiotic algal bacterial balance will be unstable during their co-cultivation as related in Arranz, A; Bordel, S; Villayerde, S; Zamarreñ, J M; Guieysse, B; Muñoz, R. Modeling photosynthetically oxygenated biodegradation processes using artificial neural networks. J. Hazard Mater., 2008, 155, 51-57 and [12].

Typically, in a closed system, the mixture of microalgae and bacteria is placed in a stirred reservoir of a reactor where microorganisms can be co-cultured as free cells [13] or as cells immobilized into various solid carriers preventing them from being washed off during industrial wastewater treatment [8]; [11]; [17].

It is also known to improve the oxygen supply for aerobic biodegradation of toxins by microbial communities by using phototrophic bio-films. The bio-films typically contain photoautotrophs, chemoautotrophs and heterotrophs, attached to a solid support and are used for effective aerobic wastewater treatment [14]; [21]. In addition to oxygen supply to bacterial partners, microalgae might remove xenobiotics from polluted environments. Algal biomass produced during photosynthetic oxygenation can further be used for agriculture, bio-diesel production and pharmaceutical applications, thus reducing cost of wastewater treatment [3]; [6]; [18]; [19]. Although photosynthetic microorganisms provide efficient, low-cost and safe tools to enhance oxygen supply for the aerobic biodegradation of waste materials, further efforts towards optimization of algal bacterial consortia are needed for the industrial application of this technology.

The present invention thus utilizes a novel approach for treatment of contaminated water. In this connection, it should be noted that, for the purposes of the present application, the term treatment should be interpreted as “processing” because, in some embodiments of the invention such processing is an actual treatment of water to produce purified water (i.e. water that has been physically processed to remove at least some of the impurities including chemical contaminants), and in some embodiments such processing may include, alternatively to the “actual treatment” or in addition thereto, production of a biomass.

The invention provides a hybrid reactor, i.e. a reactor containing at least two different species (e.g. heterotrophic and phototrophic microorganisms), for treating contaminated water (e.g. wastewater detoxification) and for producing algae biomass.

As used in this specification and the accompanying claims, the term “Wastewater detoxification” indicates reduction of a level of one or more contaminants in water to an acceptable level.

The invention utilizes aerobic heterotrophic microorganisms, such as aerobic bacteria, which break down (cause decomposition of) organic contaminants, utilizing oxygen produced by phototrophic (e.g. photosynthesizing) microorganisms.

As used in this specification and the accompanying claims, the term heterotrophic microorganisms indicates organisms requiring complex organic compounds of nitrogen and carbon for metabolic synthesis.

As used in this specification and the accompanying claims, the term phototrophic microorganisms indicates photosynthetic organisms including, but not limited to, green and purple bacteria, certain algae and photosynthetic bacteria.

Conventionally, reactors of the kind specified contain different species of microorganisms, cultivated as free or immobilized cells in a single compartment. The reactors for wastewater detoxification are based on co-cultivation of aerobic microorganisms and oxygen producing microorganisms, which share a single compartment where the aerobic microorganisms break down organic contaminants and utilize oxygen produced by photosynthesizing microorganisms.

Photosynthesizing microorganisms are highly sensitive to toxic pollutants as compared to aerobic microorganisms. It should be understood that the selection of toxin-resistant photosynthesizing microorganisms is a difficult task. Moreover, the photosynthesizing microorganisms and the aerobic microorganisms have different metabolism and growth rate. Therefore, in single-compartment reactors, the photosynthesizing microorganisms are typically injured by both the aerobic microorganism metabolism and the toxic pollutants.

In some embodiments of the invention, the phototrophic microorganisms are used including photosynthesizing microorganisms producing oxygen when illuminated.

The present invention provides an efficient, low-cost and ecologically safe technique for industrial wastewater detoxification as well as for simultaneous production of ecologically pure photosynthesizing microorganism biomass for different industrial applications, using a novel hybrid reactor. The present invention therefore provides aerobic biodegradation of waste materials, and mass production of photosynthesizing microorganisms (e.g. microalgae).

There is provided, according to one broad aspect of the invention, a reactor for treating contaminated water, the reactor comprising: a first compartment for interaction therein between the water and at least one species of aerobic heterotrophic microorganism which are adapted to break down one or more organic contaminants in said contaminated water, and a second compartment for interaction therein between the water and at least one species of phototrophic microorganism capable of producing oxygen, the two compartments interfacing one another by a gas-permeable membrane configured and operable to prevent transfer of the organic contaminant therethrough while allowing gas diffusion therethrough, thereby enabling passage of oxygen, produced by the phototrophic microorganism in the second compartment, into the first compartment to facilitate the breakage down of said one or more contaminants of the contaminated water in the first compartment.

In the present invention, the cultivated heterotrophic and phototrophic microorganisms are separated by a gas-permeable membrane. The latter is configured to prevent transfer therethrough of the organic contamination, while allowing diffusion of oxygen therethrough.

In particular, aerobic bacteria are separated from oxygen-producing microalgae by a gas-permeable hydrophobic or hydrophilic membrane. Such a membrane prevents the contamination of the second compartment (e.g. algal compartment) by organic contaminants (e.g. toxic chemicals), and allows diffusion (i.e. in a free space propagation) of oxygen from the second compartment (e.g. algal compartment) to the first compartment (e.g. bacterial compartment), CO₂ diffusion (produced by the heterotrophic microorganisms) in the opposite direction following the photosynthesizing microorganisms mass production. It should be noted that the O₂ produced by algae is used by the bacteria for their metabolic activity (e.g. respiration and various cell function), and that the CO₂ produced by the bacteria is used by algae for their cell metabolic needs (e.g. photosynthesis).

Using the reactor of the present invention prevents the atmosphere from being contaminated by hazardous pollutants during mechanical aeration. Moreover, the microorganisms may be immobilized within the reactor by using nontoxic and reusable matrices. Alternatively, the microorganisms may be freely cultivated within the reactor.

Moreover, in some embodiments, the source of light associated with the photosynthesis process may be the sunlight energy and the matrices for cell immobilization may be low-cost polymer matrices, thus reducing the costs associated with the manufacturing of the reactor. A harvesting process of microalgae is used for the reduction of algae biomass, i.e. for maintaining an optimal algae density for maximal O₂ production. The harvested removed algae biomass may be used for different industrial applications.

The reactor of the present invention may be a closed/open system and is not limited to any microorganism species, immobilization matrices, membranes, light sources or volume shape and size.

The photosynthesizing microorganisms are preferably algae, in particular unicellular photosynthetic eukaryotic or prokaryotic organisms. The latter may include but are not limited to such organisms as Chlorella, Spirolina, Dunaliella or Nannochloropsis including extremophiles (e.g. thermophile, Acidophile, Alkaliphile, Halophile, or generally organisms that thrive in or require physically or geochemically extreme conditions detrimental to the majority of life on the Earth). The photosynthesizing organisms producing oxygen may be also photosynthetic multicellular algae. It should be noted that when the reactor is configured as an open system, the cultivation of extremophilic microalgae prevents the contamination of the algal compartment by other organisms that cannot tolerate such extreme environments. The compartment containing aerobic microorganisms may include bacteria, fungi, yeasts and microalgae.

There is also provided a method for treating contaminated water. As indicated above, for the purposes of the present application, the term treatment signifies purification and/or production of a biomass.

The method comprises: providing first and second interactions of the water to be treated with respectively aerobic heterotrophic and phototrophic microorganisms, said phototrophic microorganisms enabling production of oxygen and said aerobic heterotrophic microorganisms enabling breaking down contaminants in the water, said first and second interactions being provided in different compartments being in a communication with one another preventing interaction between the contaminants and the phototrophic microorganisms while enabling gas passage in between them, thereby enabling at least one of the following: (i) involvement of oxygen, produced by said phototrophic microorganisms, in said breaking down of the contaminants; and (ii) diffusion of CO₂, produced by said heterotrophic microorganism, towards the phototrophic microorganisms.

In some embodiments, the method comprises illuminating the water under treatment to enable photosynthesis process in the phototrophic microorganisms inducing oxygen production which diffuses towards the aerobic heterotrophic microorganisms. The heterotrophic microorganisms produce CO₂ diffusing towards the phototrophic microorganisms enabling the simultaneous production of photosynthesizing microorganisms.

The method may comprise harvesting the photosynthesizing microorganisms. The microorganisms are cultivated as free microorganisms or as immobilized microorganisms. By using the technique of the present invention, the organic contaminants are aerobically biodegraded preventing contamination of atmosphere during mechanical aeration.

According to another broad aspect of the present invention, there is provided a for producing photosynthesizing microorganism biomass, the method comprising: providing first and second interactions of water with respectively phototrophic microorganisms and aerobic heterotrophic microorganisms, while illuminating the phototrophic microorganisms with light inducing a photosynthesis process therein, thereby causing breaking down contaminants in the water producing CO₂ and causing interaction of the with said phototrophic microorganisms resulting in the photosynthesizing microorganism mass production.

### BRIEF DESCRIPTION OF THE FIGURES

In order to understand the invention and to see how it may be implemented in practice, and by way of non-limiting example only, with reference to the accompanying drawing, in which

FIG. 1 schematically illustrates an example of a vertically oriented reactor; and;

FIG. 2 schematically illustrates an example of a horizontally oriented reactor.

### DETAILED DESCRIPTION OF THE EMBODIMENTS

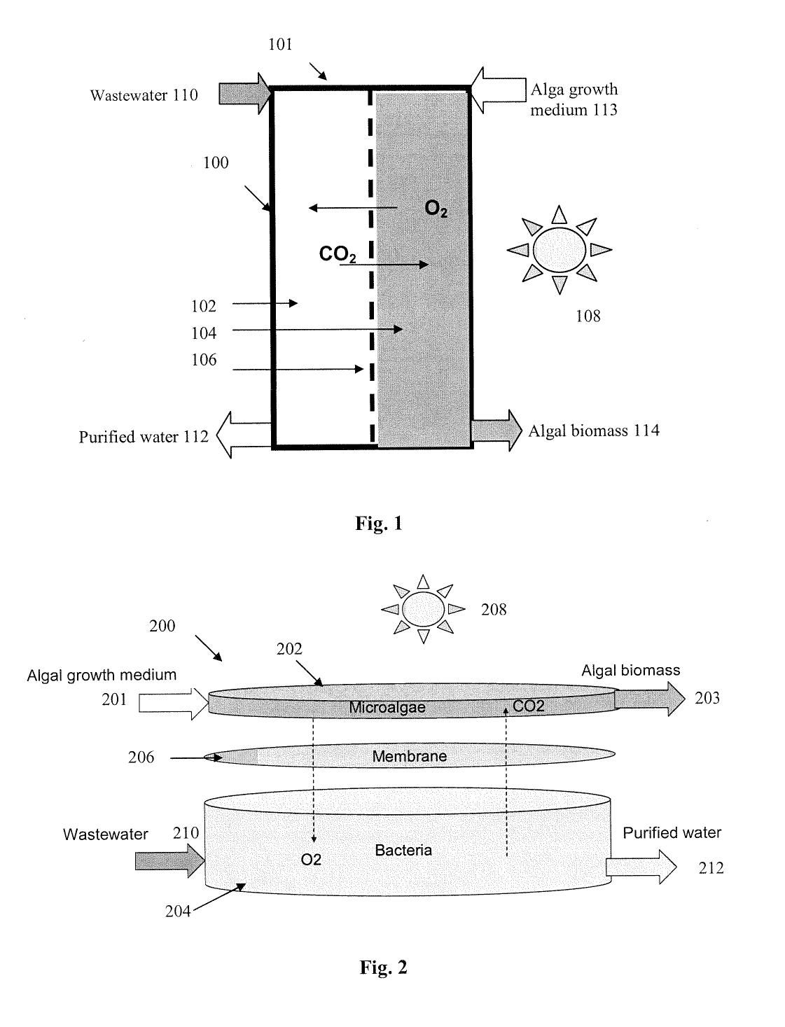

Reference is made to FIG. 1 illustrating an example of a reactor according to the invention for treating contaminated water 100. In this specific but non-limiting example, the reactor 100 has a closed configuration (i.e. unexposed to or screened from the environment) by using a cover 101. It should, however, be understood that by removing the cover, the reactor becomes of the open configuration. Also, in the present example, the reactor is intended for vertical orientation during the operation.

The reactor is configured as a so-called hybrid reactor, i.e. enables reactions involving at least two different species. More specifically, the reactor 100 comprises two compartments: one compartment 102 for containing one or more species/kinds of heterotrophic aerobic microorganisms (e.g. bacteria, fungi, yeasts, microalgae) which break down organic contaminants in wastewater, and another compartment 104 containing one or more species/kinds of phototrophic microorganisms (proliferating) producing oxygen (e.g. with evolvement of microalgae biomass).

A gas-permeable membrane 106 is provided as an interface between the two compartments 102, 104. Such a membrane 106 is configured and operable to prevent passage of contaminants (toxic chemicals) of the contaminated water (wastewater) from compartment 102 to compartment 104 thus preventing contamination of the algal compartment 104, and allows diffusion (passive or forced) in a free space of propagation of oxygen from the algal compartment 104 to the bacterial compartment 102, and in some embodiments, also allows CO₂ diffusion in the opposite direction allowing the photosynthesizing microorganism mass production (e.g. supporting algal photosynthesis).

In some embodiments, the reactor 100 is associated with a light source 108 which enables the photosynthesis process of the phototrophic microorganisms in which the microorganisms convert carbon dioxide (produced by the heterotrophic microorganisms) to oxygen when illuminated. The heterotrophic microorganisms produce CO₂ which diffuses from compartment 102 to compartment 104 allowing the photosynthesizing microorganism mass production. The oxygen then diffuses through gas-permeable membrane 106 from the compartment 104 towards the compartment 102, and the heterotrophic microorganisms break down organic contaminants in the contaminated water by utilizing the oxygen produced by the phototrophic microorganisms.

The light source 108 may be of the kind utilizing sunlight, or that utilizing continuous or intermittent artificial light (e.g. a luminescent lamp). Although the present example as illustrated in the figure utilizes sunlight radiation, it should be understood that the invention is not limited to this specific example, and the reactor of the present invention can be used with any light source. The light source may be associated with reflector(s) to controllably direct the illumination onto the reactor. In a non-limiting example, the optimal wavelength range of light used may be about 400-700 nm.

In the photosynthesis process, the light energy is converted into chemical energy by the photosynthesizing microorganisms. The initial substrates are carbon dioxide and water, and the end-products are oxygen and carbohydrates constituting a part of the alga biomass. The membrane may be a hydrophobic gas-permeable membrane made from silicon, polyethylene etc., as illustrated in the table below from Biotechnol. Prog. 2005, 21, 741750.

TABLE 1
Properties of Membrane Candidates for Gas-Liquid Separation

| | | | gas permeability ( [symbol] | |
|---|---|---|---|---|
| polymer | structure | thickness (μm) | O₂ | CO₂ |
| polyethylene | dense | 145 ± 3 | 8.5 × 10⁻¹² | 3.2 × 10⁻¹¹ |
| | | 149 ± 4 | 7.5 × 10⁻¹² | 3.0 × 10⁻¹¹ |
| low-density | dense | 26 ± 3 | 4.7 × 10⁻¹¹ | 1.3 × 10⁻¹⁰ |
| polyethylene | | 28 ± 2 | 6.1 × 10⁻¹¹ | 1.5 × 10⁻¹⁰ |
| BioFOLIE^{a} | dense | 22 ± 1 | 9.5 × 10⁻¹⁰ | 1.1 × 10⁻⁹ |
| | | 21 ± 1 | 9.7 × 10⁻¹⁰ | 1.3 × 10⁻⁹ |
| polypropylene | porous | 25 ± 2 | NM^{b} | NM |
| | | 26 ± 3 | NM | NM |
| PTFE | porous | 54 ± 1 | NM | NM |
| | | 57 ± 1 | NM | NM |

^{a}Composite material (Sartorius, France).
^{b}NM, not measurable (very high). [symbol]
indicates data missing or illegible when filed

Alternatively, the gas-permeable membrane can be made from hydrophilic materials (e.g. polyurethane).

Turning back to FIG. 1, the reactor 100 has inlet and output ports 110, 113, 112 and 114. Inlet and output ports 110 and 112 respectively are made in compartment 102 and serve for introducing wastewater, via inlet 110 and discharging purified water from outlet port 112. Inlet and output ports 113 and 114 are made in compartment 104 for respectively introducing alga growth medium and discharging algal biomass. Thus, in the present example, photosynthesizing microorganisms, such as algae, in particular photosynthetic eukaryotic, prokaryotic organisms, or photosynthetic multicellular algae, proliferate in large-scale, are located in compartment 104. The photosynthesizing microorganisms produce oxygen which penetrates (e.g. by diffusion) through the membrane 106 in the compartment 102 to allow aerobic microorganism to break down the contaminants of the wastewater being supplied to compartment 102. The aerobic microorganisms use oxygen as an electron acceptor, break down the organic chemicals, and produce carbon dioxide and water (purified). The purified water exits from the outlet port 112. The carbon dioxide diffuses through the membrane 106 facilitating photosynthesis of the photosynthesizing microorganisms. The algal biomass is harvested from the outlet port 114.

It should be noted that the second compartment 104 is configured and operable to enable micro-organism proliferation by combining an optimal light irradiation with an appropriate algae growth medium, a CO₂ supply, and an algae density control. It should be understood that optimal light irradiation (OLI) may be different for different alga strains. For example, the maximal rate of photosynthesis for blue-green algae may be achieved at 20 μmol photon m⁻² s⁻¹. The s⁻¹, while for Chlorella, it will be achieved at 700 μmol photon m⁻² s⁻¹. The following Table 2 exemplifies media suitable for cultivation of Chlorella algae:

TABLE 2

| | | |
|---|---|---|
| Proteose peptone (Oxoid L85)* | 1.0 | g |
| MgSO₄•7H₂O (1.0 g/L) | 20.0 | ml |
| K₂HPO₄ (1.0 g/L) | 20.0 | ml |
| KNO₃ (10.0 g/L) | 20.0 | ml |
| Water to | 1.0 | L |

The alga density may be estimated by different methods, for example electronic particle counter using Coulter counter, or microscopic methods using hemacytometer.

In this specific example, the microorganisms are freely cultivated within the reactor. However, the microorganisms may be immobilized within the reactor by using nontoxic and reusable matrices (not shown).

Reference is made to FIG. 2 illustrating another example of a reactor 200 of the present invention configured as a closed reactor intended for horizontal orientation during the operation. The reactor 200 comprises one compartment 204 containing aerobic microorganisms, and another compartment 202 containing photosynthesizing microorganisms producing oxygen (e.g. with evolvement of microalgae biomass). The reactor 200 is associated with a light source 208 (e.g. sunlight, artificial light) which enables the photosynthesis process to occur in which the microorganisms convert carbon dioxide to oxygen when illuminated.

The two compartments 202, 204 are separated by a hydrophobic or hydrophilic gas-permeable membrane 206. Such a membrane 206 prevents contamination of the algal compartment 202 by toxic chemicals from wastewater, and allows free diffusion of oxygen from the algal compartment 202 to the bacterial compartment 204, and CO₂ diffusion in the opposite direction to support algal photosynthesis.

The reactor 200 has inlet and output ports. An inlet port 210 is made in the compartment 204 for introducing wastewater into this compartment. Purified water exits compartment 204 via the outlet port 212. Growth medium enters compartment 202 via inlet 201, and algal biomass is harvested from compartment 202 via outlet port 203. Carbon dioxide diffuses through the membrane 206 facilitating the photosynthesis of the photosynthesizing microorganisms.

The reactor of the invention can thus be open or closed, may have various orientation (vertical orientation as exemplified in FIG. 1 or horizontal one as illustrated in FIG. 2), as well as various shapes (flat or tubular), volumes, wall flexibility, and weight. The reactor can have transparent part(s), e.g. an optical window, for exposing its inside to light, and may also be configured to enable access of is inside by light of specific wavelengths, e.g. by using appropriate spectral filters, e.g. external to or incorporated within the optical window.

1. A reactor for treating contaminated water, the reactor comprising: a first compartment for interaction therein between the water and at least one species of aerobic heterotrophic microorganism which are adapted to break down one or more organic contaminants in said contaminated water, and a second compartment for interaction therein between the water and at least one species of phototrophic microorganism capable of producing oxygen, the two compartments interfacing one another by a gas-permeable membrane configured and operable to prevent transfer of the organic contaminant therethrough while allowing gas diffusion therethrough, thereby enabling passage of oxygen, produced by the phototrophic microorganism in the second compartment, into the first compartment to facilitate the breakage down of said one or more contaminants of the contaminated water in the first compartment.

2. The reactor according to claim 1, wherein said aerobic heterotrophic microorganism uses oxygen produced by said phototrophic microorganism to break down organic contaminant in said contaminated water.

3. The reactor according to claim 1, wherein said phototrophic microorganism comprises photosynthesizing microorganism that produces oxygen when illuminated.

4. The reactor according to claim 1, wherein said gas-permeable membrane is configured to enable diffusion of CO₂, produced by said heterotrophic microorganism, from the first compartment towards the phototrophic microorganisms in the second compartment, thereby enabling production of photosynthesizing microorganisms in said second compartment.

5. The reactor according to claim 1, wherein said gas-permeable membrane is hydrophobic.

6. The reactor according to claim 1, wherein said gas-permeable membrane is hydrophilic.

7. The reactor according to claim 1, comprising at least one inlet port and at least one outlet port, the at least one inlet port serving for feeding said at least one species of aerobic heterotrophic microorganism and said at least one species of phototrophic microorganisms, the at least one outlet port serving for discharging purified water from said reactor.

8. The reactor according to claim 1, comprising at least one inlet port and at least one outlet port, the at least one inlet port serving for feeding said at least one species of aerobic heterotrophic microorganism and said at least one species of phototrophic microorganisms, the at least one outlet port serving for discharging a biomass, produced during said interaction between the water and the at least one species of phototrophic microorganism, from said reactor.

9. The reactor according to claim 1, having a closed configuration being separated from environment of surroundings during operation.

10. The reactor according to claim 1, having an open configuration allowing interaction with environment of surroundings during operation.

11. The reactor according to claim 1, wherein said first and second compartments contain immobilized heterotrophic and phototrophic microorganisms.

12. The reactor according to claim 1, wherein said first and second compartments contain free cultivated heterotrophic and phototrophic microorganisms.

13. The reactor according to claim 3, wherein the photosynthesizing microorganisms producing oxygen are selected from photosynthetic eukaryotic and prokaryotic organisms.

14. The reactor according to claim 3, wherein the photosynthesizing microorganisms producing oxygen are photosynthetic multicellular algae.

15. The reactor according to claim 1, wherein the aerobic microorganisms are selected from bacteria, fungi, yeasts and microalgae.

16. The reactor according to claim 3, configured to enable exposure of an interior of the second compartment to light energy.

17. The reactor of claim 16, comprising an internal light source accommodated to direct light into the second compartment.

18. The reactor of claim 16, wherein the second compartment comprises an optical window exposed to the light energy.

19. A method for treating contaminated water, the method comprising: providing first and second interactions of the water to be treated with respectively aerobic heterotrophic and phototrophic microorganisms, said phototrophic microorganisms enabling production of oxygen and said aerobic heterotrophic microorganisms enabling breaking down contaminants in the water, said first and second interactions being provided in different compartments being in a communication with one another preventing interaction between the contaminants and the phototrophic microorganisms while enabling gas passage in between them, thereby enabling at least one of the following: (i) involvement of oxygen, produced by said phototrophic microorganisms, in said breaking down of the contaminants; and (ii) diffusion of CO₂, produced by said heterotrophic microorganism, towards the phototrophic microorganisms.

20. The method of claim 19, comprising illuminating the phototrophic microorganisms to enable a photosynthesis process to occur in said phototrophic microorganisms thereby inducing production of oxygen which diffuses towards said aerobic heterotrophic microorganisms.

21. The method of claim 20, wherein said heterotrophic microorganisms produce CO₂ diffusing towards said phototrophic microorganisms enabling the simultaneous production of photosynthesizing microorganisms.

22. The method of claim 21, comprising harvesting said photosynthesizing microorganisms.

23. The method of claim 19, wherein the heterotrophic and phototrophic microorganisms are cultivated as free microorganisms.

24. The method of claim 19, wherein the heterotrophic and phototrophic microorganisms are cultivated as immobilized microorganisms.

25. The method of claim 19, wherein said organic contaminants are aerobically biodegraded preventing contamination of atmosphere during mechanical aeration.

26. The method of claim 19, comprising producing purified water resulted from said interaction between the aerobic heterotrophic microorganisms and the water under treatment.

27. The method of claim 20, comprising producing alga biomass as a result of conversion of light energy into chemical energy by the photosynthesizing microorganisms.

28. A method for producing photosynthesizing microorganism biomass, the method comprising: providing first and second interactions of water with respectively phototrophic microorganisms and aerobic heterotrophic microorganisms, while illuminating the phototrophic microorganisms with light inducing a photosynthesis process therein, thereby causing breaking down contaminants in the water producing CO₂ and causing interaction of the with said phototrophic microorganisms resulting in the photosynthesizing microorganism mass production.

29. The method of claim 28, wherein said first and second interactions are provided in different compartments being in gas communication between them allowing oxygen, produced as a result of said first interaction, to be used in said second interaction and allowing the CO₂ produced as a result of the second interaction to be used during said photosynthesis process.
